(19) United States
(12) Patent Application Publication
(10) Pub. No.: US 2015/0122878 A1
(43) Pub. Date: May 7, 2015

(54) USING TRACER TECHNOLOGY TO IDENTIFY PRODUCTION DETAILS

(71) Applicants: Scott J. Collick, Lake Jackson, TX (US); Gregory M. Bunker, Midland, MI (US); Thomas Allgeuer, Wollerau (CH); John W. Garnett, IV, Omaha, NE (US); Dana R. Breed, Lake Jackson, TX (US)

(72) Inventors: Scott J. Collick, Lake Jackson, TX (US); Gregory M. Bunker, Midland, MI (US); Thomas Allgeuer, Wollerau (CH); John W. Garnett, IV, Omaha, NE (US); Dana R. Breed, Lake Jackson, TX (US)

(73) Assignee: DOW GLOBAL TECHNOLOGIES LLC, Midland, MI (US)

(21) Appl. No.: 14/073,061

(22) Filed: Nov. 6, 2013

(51) Int. Cl. G01N 33/44; G06K 19/02

(52) U.S. Cl. CPC G01N 33/442; G06K 19/022
USPC 235/375; 252/408.1

(57) ABSTRACT

The present invention is a method of tagging resin product such that it can be identified. The method involves adding a series of tracer elements to a resin in specific amounts. The relative amounts of one or more of such tracer elements for each desired unit of production are varied, such that each unit of production has a unique combination of ratios of the various tracer elements.

### FIELD OF THE INVENTION

The present invention relates to a method of tagging let numbers of polymer production so that resin obtained in the marketplace can be analyzed and the source and lot numbers identified.

### BACKGROUND AND SUMMARY OF THE INVENTION

Suppliers of resin, or intermediate articles made from resin, frequently would like to be able to identify the source or even the particular lot numbers which a product was made. For example, manufacturers often promote the use of their materials by providing manufacturing equipment. Thus, a film supplier may provide and end-user with equipment used to convert film to a pouch or other package. This equipment and related technology may be provided at no cost, or at a reduced royalty rate, for so long as the end-user purchases all of its raw materials (film in this example) from the supplier who provided the equipment. Additionally, if quality control issues arise, the seller of such materials may wish to identify which lot number is involved so that similar materials can be identified before causing additional problems.

Currently, however, there is no reliable way to ensure that the end-user is using only raw materials obtained from the supplier that provided the fabrication equipment, or providing accurate information as to how much alternatively-sourced raw materials are being used, or ensuring that only approved raw materials have been used with the equipment, or to be able to identify not only the source of the material but the particular day or time such material was produced.

Technology now exists to allow resin manufacturers to add minor amounts of a unique material, sometimes referred to as a tracer element, to a resin. The tracer element is chosen so that it does not interfere with the performance of the resin, but can be detected using routine analysis. Such technology includes the FINGERPRINT™ Resins available from The Dow Chemical Company. Such modified resins are currently used to enable the resin manufacturer to evaluate and improve quality and consistency of the resins when used in applications not in the resin manufacturer's control. Thus, for example, Dow can get performance data on its resins by obtaining samples of pipe in actual use, if that pipe contains the tracer element which is unique to Dow.

The present invention involves a new use for this existing tracer element technology. The present applicant has found that the tracer technology can also be used to ensure that raw materials used with a piece of fabrication equipment come from an approved source, or at least monitor and report the amounts of alternatively-sourced raw material. The method involves the step of including an on-line analyzer with the equipment. The on-line analyzer will be capable of detecting a tracer element which can be included in the raw material being supplied for use with the equipment. If the tracer element is not detected in the raw materials being used with the fabrication equipment, then the fabrication equipment can be programmed to shut down. Alternatively, the fabrication equipment can be programmed to record the amounts of material processed which did not include the tracer element. In this way an accurate record of how much alternatively-sourced material is being used can be obtained, and appropriate royalties can be collected.

This technology can conceivably be used in multiple steps of a process to make an article of commerce. A tracer element or series of tracer elements can be added to the resin itself as part of the polymerization reaction or shortly afterwards as part of the pellitization process. Film, fiber, sheets or finished articles made from that resin will then also contain the tracer element(s). Alternatively the tracer element(s) may be able to be added at later stages such as in film or fiber production.

### DETAILED DESCRIPTION OF THE INVENTION

In one embodiment, the present invention is a method of tagging resin product such that it can be identified. The method involves adding a series of tracer elements to a resin in specific amounts. The relative amounts of one or more of such tracer elements for each desired unit of production are varied, such that each unit of production has a unique combination of ratios of the various tracer elements.

This technology can also be used for ensuring that raw material used with a particular piece of fabrication equipment comes from an approved source. The method involves first providing fabrication equipment to a user of the raw material. The fabrication equipment will include an on-line analyzer for detecting the presence of a series of one or more tracer elements. The on-line analyzer will be in electronic communication with the fabrication equipment to safely shut down the equipment in the event that raw material which does not contain a specified combination of tracer element in a specified amount is attempted to be used with the fabrication equipment. The details of the identity and characteristics of the tracer element should be provided only to those raw material suppliers who have demonstrated that their products meet the requisite quality control standards. Thus only raw materials which have been pre-approved by the equipment manufacturer will contain the specific tracer element(s) in specific amount. Accordingly, only approved raw materials will be capable of being processed by the fabrication equipment.

The specifics of the fabrication equipment are not limited in the present invention. In theory any fabrication equipment which converts raw material into a different product can be used with the present invention. It should be understood that “raw material” includes resin itself, but also includes films, fibers, sheets, and articles of any kind which may be used in a piece of fabrication equipment to be converted to another product in some manner. This includes packaging units which are “converted” only in the sense of being converted from an empty package to a full package; that is, the empty package is converted in the sense of being filled and sealed.

In another embodiment, the present invention is a method for increasing sales of a raw material. Often the fabrication equipment represents a hurdle for manufacturers, often just due to expense, but occasionally because the equipment may be protected by intellectual property rights. Raw material suppliers may be in a position to license, sell or lease the equipment to overcome this barrier. The raw material supplier may even do this below the market rate, in exchange for the manufacturer's assurance that they will purchase raw material for use in the equipment from the raw material supplier who helped in providing the equipment. In such a case, the on-line analyzer will be in electronic communication with a means for recording the specific amounts of raw material used which does not contain the tracer element. This information can be used to allow the equipment supplier to charge royalties, or otherwise seek compensation for the cost of the equipment and/or technology.

The tracer element for use with the present invention can be any tracer material known in the art. In general the tracer element can be any chemical which can be detected by a non-destructive analytical method like spectroscopy (including techniques such as IR, NMR, x-ray, etc) and which is not commonly used as an additive for the polymers/applications. In other words, the tracer element should be distinguishable from other additives which may likely be included in a polymer formulation (for example, antioxidants etc.) but still sufficiently “hidden” so that potential counterfeiters can not easily identify the substance used as the tracer element and include it in their own polymer formulations in order to pass off their formulations as coming from the original source.

These tracer elements include materials of organic origin like polystyrene and polypropylene (depending on the raw material), and/or materials of inorganic origin like metal-complexes/metal salts. Metal complexes/salts of transition metals like Niobium (Nb), Tantalum (Ta) and Molybdenum (Mo) can be advantageously used for certain raw materials. Examples include: alkylidene complex of Nb and Ta, Niobium nitrate nano particles (superconductor), Niobium-2-ethylhexanoate (yellow liquid soluble to organic solvents), Niobium alcoholate clusters, and etoxi with metal (M[Nb(OEt)6]2 where M═Mg, Ca, Sr, Ba). Rare earth elements could be also used as tracers, like oxides of Yttrium, Ytterbium, Erbium, Neodymium or Europium.

Other commonly known tracers capable of being used in the present invention include nano-particles and/or commonly known biomarkers (pristane, phytane, steranes, triterpanes, porphyrin, etc)

The tracer material should not interfere with the intended use of the raw material. Thus for example, only tracer elements which are certified for use with food should be used in any raw material which is intended for use in packaging food products. The selection of the tracer element should also take into account the steps in the manufacturing process with the raw material is expected to undergo. For example if it is known that particular resin pellets will be spun into a fiber, then tracer element of very small particle size would be desired so as to not interfere with the spinning process. Similarly, if a particular resin will be compounded with other resin at high temperatures and pressures to ensure proper mixing, then the tracer element should be selected to ensure that it will not degrade at such temperatures or pressures.

It should also be understood that more than one tracer element can be added. Particularly if more than one raw material supplier is adding tracer elements to their products, a combination of two or more tracer elements may be desirable to provide unique identifiers. In certain embodiments 4, 6 or even more tracer elements can be added to the resin. Over the course of production each of the tracer elements may be adjusted to be omitted, or to be added in a low medium or high amount. Thus for example, a manufacturer may determine that it will include six tracer elements, A-F, as taggants in order to code each unit of production. Each taggant may then be varied in an amount for example 0, 10, 20, 30 or 40 ppm. Thus a first unit of production may be coded as A-40, B-20, C-20, D-10, E-0, and F-30. As this resin is blended with other materials the ratios of these taggant materials will remain the same, allowing the code to be consistent. By using more and more taggants and differing the amounts of each taggant, a nearly infinite number of unique codes could be generated.

Thereafter, any material in the marketplace or at a customer having quality control issues can be analysed to identify the presence of the tracer elements and the ratios of each element to one another. The result of such analysis can then be compared to the production record, to identify the particular unit of production (i.e. lot number).

The analyzer can be chosen from among any of those commercially known to be capable of detecting the tracer element (or elements) selected to include in the source of the raw material. These include chemical analysis, electro-magnetic techniques, NMR or photochemistry/optical analysis, on-line gas chromatography and spectroscopy.

For purposes of this invention, “on-line” analyzers include analyzers which are analyzing material as it is proceeds along the manufacturing process, as well as analyzers which remove small amounts of material from the manufacturing process for analysis. These latter types of analyzers are sometimes referred to as “at-line” detectors. It should be understood that the analyzer is not required to be on-line or at line where the present invention is being used to tag and indentify particular lot numbers.

What is claimed is:

1. A method of tagging resin product such that it can be identified comprising the steps of
a. adding a series of tracer elements to a resin in specific amounts;
b. varying the relative amounts of one or more of such tracer elements for each desired unit of production, such that each unit of production has a unique combination of ratios of the various tracer elements.

2. The method of claim 1 wherein there are 6 or more tracer elements which can be added to the resin.

3. The method of claim 2, wherein each tracer element may be omitted or be added at a low, medium or high amount.

4. A method of identifying a particular unit of production in which a resin product was made comprising the steps of:
a) producing a resin;
b) adding a series of tracer elements to the resin in specific amounts to produce a tagged product;
c) adjusting the amounts of one or more of the tracer elements for each unit of production, such that each unit of production contains a unique combination of tracer elements in specific ratios to each other;
d) keeping a record of each unique combination of tracer elements in specific ratios and any desired production data associated with that unit of production;
e) analyzing a material which may contain the tagged product to identify the presence of the tracer elements and the ratios of each element to one another;
f) comparing the identity of the tracer elements and the ratios of the amounts of the tracer elements to the record, to identify the particular unit of production.

5. The method of claim 4 wherein there are at least 6 different tracer elements which may be added to the resin.

6. The method of claim 5, wherein each tracer element may be omitted or be added at a low, medium or high amount.
